(19) United States
(12) Patent Application Publication
(10) Pub. No.: US 2003/0232114 A1
(43) Pub. Date: Dec. 18, 2003

(54) Method for liquid enrichment with oxygen and applications of enriched liquids

(76) Inventor: Nikola Dekleva, Belgrade (YU)

Correspondence Address: James V. Costigan, Esq., HEDMAN & COSTIGAN, P.C., 1185 Avenue of the Americas, New York, NY, 10036, US

(21) Appl. No.: 10/171,171

(22) Filed: Jun. 13, 2002

(51) Int. Cl. A61K 33/00; A23C 1/00; A23B 4/00

(52) U.S. Cl. 426/312; 424/600

(57) ABSTRACT

The present invention relates to a method for preparing oxygen enriched liquids by subjecting a liquid under hyperbaric oxygen conditions within an oxygen enrichment chamber of a sealed hyperbaric apparatus and maintaining the liquid under hyperbaric pressure in conditions and for a time sufficient to dissolve and stabilizing the oxygen in the liquid. The oxygen enriched liquids are suitable for use in cosmetic and medical field and for preparing a drinkable oxygen enriched beverage.

### FIELD OF THE INVENTION

The present invention relates to oxygen enriched liquids, methods for making the same and their applications in the medical and cosmetic fields.

### BACKGROUND OF THE INVENTION

It is known that mammals are not capable of making their own oxygen reserve and therefore an oxygen deficiency in the body often causes tissues changes known as hypoxia, hypoxidosis and anoxia.

Transport of the oxygen is linked to blood vessels, erythrocytes, hemoglobin, iron and DPG. Since erythrocytes do not enter the cells, diffusion process occurs, enabling the penetration of oxygen through the cell membrane, causing the cell metabolism to function and creating electrical potential of the cells, as a manifestation of life of those cells.

Every obstacle such as thrombi or injury that led to strangulation or obstacle to the vessel circulation is preventing the oxygen to supply the tissue.

The deficiency of molecular oxygen in the tissue is imperiling the tissue and pathological changes occur. These pathological changes in pathology and patho-physiology are classified from anemia to gangrene.

To date numerous methods have been developed to attempt to improve oxygen delivery to tissues.

The law of physics of Daltons, Henry, Gay-Lussac, have proved that every gas dissolves in liquids when put under pressure, which means that with higher pressure the solubility of the gas grows in the liquid. According to this principle, we have all the gasses dissolved in our body in liquid form.

Thus in the past numerous methods have been developed to improve oxygen delivery to tissues by means of inhalation through devices such as tents, cannulas, masks, hoods and mechanical ventilator systems.

The purposes of oxygen inhalation is to increase the amount of oxygen absorbed by erythrocytes to increase oxygen delivery to cells and tissues. However the inhalation systems of oxygen are associated with several drawbacks and complications.

In the attempt to overcome these limitations, Prof. Boerema et al. performed the usage of increased oxygen dissolved in liquids as disclosed in “Life without blood: A study of the influence of high atmospheric pressure and hypothermia on dilution of blood”; J. Cardiovas. Surg.; 1, 133 1960. This article represents the beginning of the usage of oxygen under pressure or dissolved oxygen under pressure in all liquids of the human organism. The disclosed method of usage under pressure is widely spread today and it is used as a routine in the hyperbaric medicine. According to the disclosed practice, patients within a hyperbaric chamber inhale the oxygen through an oxygen mask under the simulated conditions of depth.

If the obstacle of oxygen transport in the tissue exists, this obstacle is superseded by the physically dissolved oxygen, both in the bigger blood vessel and in the microcirculatory net.

Being dissolved in all the liquids of the organism, physically dissolved oxygen is being consumed 20% or 4 times more efficiently then inhaled through the mask in the hyperbaric chamber.

However, many limitations exists for these methods of oxygen therapy.

Firstly, inhaled in the hyperbaric chamber the consumption of oxygen is only 5% and the increased blood oxygen levels achieved during chamber pressurization are lost when the chamber is depressurized and the patient is removed from the chamber.

Secondly, numerous complications have been associated with the use of hyperbaric breathing chambers including fires, explosions, oxygen toxicity, gas-embolism and diseased caused by a rapid depressurization.

The occurrence of these drawbacks have led to limit the use of hyperbaric chambers only to a restricted number of conditions.

Thus, suggestions have been made in the prior art to achieve an alternative way to increasing the amount of oxygen available for aerobic and therapeutic processes.

Other methods of oxygen therapy involves the intravenous injection of gaseous oxygen or the use of solutions oxygenated by means of a complicate gas liquid contact apparatus. These methods have been found too hazardous or too complicated.

Thus recently have been developed some methodologies to obtain a sufficiently high degree of oxygen enrichment of water.

It is also known to impregnate at elevated atmospheric pressure with a gas from an external source a substantially gas-free liquid in order to make the liquid effervescent. This artificial impregnation of a liquid is carried out by bubbling the gas through the liquid or by spraying the liquid into the gas or by violently agitating the liquid in the presence of a gas.

However, these methods have not been found to be successful to obtain liquids at high rate of dissolved oxygen or to retain the dissolved oxygen in the liquid for a prolonged time.

U.S. Pat. No. 5,766,490 in the name of Taylor et al. discloses a process for inserting and dissolving oxygen in water in an oxygen enriching space by passing a liquid and the oxygen through static and dynamic turbulent mixers and recovering the oxygenated liquid.

A drawback of the disclosed process is that it requires a complicated apparatus for enriching water with oxygen. In addition the produced oxygen enriched water leads to bubbles appearance and therefore the disclosed method is unsuitable to retain the dissolved oxygen in the water for an appreciable length of time.

U.S. Pat. No. 5,814,222 in the name of Zelenák et al. describes a method for enriching a liquid with oxygen and an apparatus including a vessel provided with a number of horizontal trays vertically spaced apart from each other which allow to dissolve the supplied oxygen in a liquid. The liquids oxygenated by the disclosed apparatus are used in various aerobic and therapeutic processes. The disclosed invention has the disadvantage that it is very difficult to maintain a desirable level of cleanness and sterility of the disclosed complicated apparatus. Accordingly, the oxygenated liquids prepared with the disclosed invention are not useful for medical purposes where strictly requirements of sterility are required.

EP 0 847 959 A1 discloses a method for producing liquids and beverages enriched with oxygen by supplying oxygen under pressure to a liquid within a bottle prefilled with oxygen. The disclosed liquid is useful only for the oral administration as oxygen integrator.

However, the oxygen incorporated in these liquid is not stable under warm temperatures and tends to lose the additional oxygen loading when the bottle are mixed such as during transport.

Thus, none of the known methodologies for increasing the oxygen content in liquids have proved successful to achieve a sufficiently degree of oxygenation of the liquid and to maintain this feature for an appreciable length of time.

Accordingly at present there is a need for increasing the content of pure oxygen in water or liquids in general to achieve a high level of dissolved oxygen and to retain the dissolved oxygen for a prolonged time.

### SUMMARY OF THE INVENTION

It is a principal aim of the present invention to provide a method for producing an oxygen enriched liquid which retain the dissolved oxygen at high concentration for an appreciable length of time.

Another object of the present invention is to enable production of high oxygen levels dissolved in water with a good retention of the dissolved oxygen even after storage, at substantially reduced costs.

Yet another object of the present invention resides in providing therapeutic or cosmetic methods of treatments employing physiological liquids enriched with oxygen in accordance with an aspect of the present invention.

A further object of the present invention is to provide a highly oxygen enriched drinkable water or beverage useful as oxygen diet-supplement.

Still another object of the present invention is to provide a method of oxygen therapy which is safe and avoids complications and drawbacks associated with method of prior art.

Essentially, the method of oxygen enrichment according to a first aspect of the present invention involves subjecting a liquid under hyperbaric and specific conditions for a time sufficient to achieve a stable enriched oxygen liquid.

Thus, in accordance with a first aspect of the present invention a method for enriching a liquid with oxygen is provided comprising introducing a liquid into a sealed container and dissolving oxygen in said liquid under pressure and recovering the liquid enriched with oxygen.

As used throughout the specification and claims the term liquid is meant to include any liquid that is capable of being oxygenated under pressure such as water, physiological solutions, flavored or mineral waters and liquid preparations for medical uses.

The method according to one aspect of the present invention does not impart any taste to the resulting liquid thus permitting the use of the water enriched with oxygen as a supplement liquid in the diet.

Typically, the sealed enriching container used in method of the invention is mechanically cleaned and disinfected before the liquid treatment. Advantageously, the enriching container is made of stainless steel and comprises a sealed chamber that supports high pressure such as 150 atmospheres.

The oxygen dissolved in the liquid under pressure preferably consists of clean medical oxygen, a very pure oxygen used in the medical field.

In particular the use of 100% pure medical oxygen in accordance with the pharmacopoeia provide oxygen enriched liquids useful for the administration to humans in need of a therapeutic or prophylaxis treatment.

In accordance with an embodiment of the invention, the liquid to be treated is firstly supplied into the sealed stainless steel chamber and then an oxygen flow is supplied in said chamber such that the oxygen dissolves in the liquid. The interior of the chamber is put under pressure for producing a high oxygen loading of the liquid and to obtain a sufficiently high degree of oxygen enrichment of water which allows to retain the dissolved oxygen in the liquid for an appreciable length of time under atmosphere pressure. Preferably the liquid is subjected to the superoxidation procedure for a time between 10 and 90 minutes, more preferably for a time between 50 and 70 minutes. After the exposition time to high pressure is terminated, a decompression period begins until the atmosphere pressure is achieved. When the atmosphere pressure is achieved, then the oxygen enriched liquid can be taken out the chamber and collected in recipients for storage.

In accordance with an embodiment of the invention the liquid within the enrichment chamber is subjected to an oxygen pressure of 3 to 40 atm, preferably of from 8 to 12 absolute atmospheres, conveniently for a time within the range of 45 to 80 minutes.

In these conditions, the process of the invention enables the dissolution of at least 30 mg/l oxygen in the liquid and the maintenance of the achieved oxygen levels in the liquid for a prolonged time under common storage conditions.

In accordance with another embodiment of the present invention, a method to enrich oxygen which allows to substantially avoid the formation of bubbles from the gas dissolved in said oxygen is provided in which oxygen is dissolved into a liquid preferably under a partial pressure of 3 to 10 Mpa (30-100 Bar) and then the oxygen enriched liquid is passed through capillary tubes.

Specifically, in order to prepare an oxygen enriched water solution for endoarterial or endovenous administration, a sterilized physiological solution is enriched with oxygen in accordance with the above disclosed methods and then said physiological solution is introduced into capillary tubes preferably made of silicon and having a diameter between 60-90μ under a hydrostatic pressure which is conveniently selected between 50-60 Mpa in order to pass with great velocity through said capillary tubes.

In accordance with a preferred embodiment, a physiological solution enriched with oxygen in accordance with the method of the invention, is supplied into silicon capillaries under hydrostatic pressure of 50-70 Mpa in order to obtain a transit of 1 g/min with the speed of 4 m/s through the capillary.

Liquid solutions which are useful for endovenous or intravenous administration include physiological solutions, dextrose solution, Ringer or Hartmann solutions, perfluoro carbonic solutions and protein solutions such as PVP plasma and alike.

In accordance with another aspect, the present invention also provides liquids enriched with oxygen obtained in accordance with the above methods.

These oxygen enriched liquids may be used as such or in a form diluted with other liquids. Suitably, these liquids have highly dissolved oxygen with standard partial pressure and with permanent bonds between the molecules of oxygen and hydrogen.

In particular, it has been found by performing depth chemical bond analyses that in the oxygen enriched water of the invention the molecules form many hydrogen bonds, resulting in a structure that resembles the structure of ice. These hydrogen bonds permit to achieve a high degree of oxygen enrichment of water and to retain the dissolved oxygen for longer period of time than with the known methodologies. In particular, the molecules of hydrogen and oxygen in the oxygen enriched water according to the invention are very close to each other to provide a stable molecular structure.

The oxygen enriched liquids of the invention are also useful in therapeutic methods of treatment of the human body or its parts, in conditions or diseases where an increased demand of oxygen is required.

Suitable therapeutic methods in which liquids made in accordance with the present invention can be advantageously employed include all the human conditions or pathologies where it is requested to increase the oxygen content of blood and tissue.

The oxygen enriched liquids can be administered either as solutions per oral use or as solutions for internal use.

In particular, water enriched with oxygen according to the invention may be orally administered for treating or preventing gastritis, ulcers, ulcus ventricle or duodena, duodenitis, colitis ulcerosa and to the pathologies where lesions at the mucous membrane are manifested. In these cases the direct application of the oxygen enriched solutions alleviates the symptoms and cures the damaged tissues permitting an oxygen transfer.

Advantageously, sterile physiological solutions enriched with oxygen according to an embodiment of the invention can be orally or parentally administered in particular to prevent or treat all cases where degenerative changes to tissues occur such as in spondilosis deforming, coxartrosis, Parkinson disease, Alzheimer disease, encephalomyelitis disseminant, stroke and other acute or chronic pathologies. In addition, in cases of vessel or circulatory insufficiency, and chronic long term damages or organs oxygen enriched solutions allow a more intensive metabolism, preventing the chronic hypoxya that appeared for any reason through the course of time.

In accordance with another feature of the invention, solutions enriched by oxygen of the invention can be intraarterially or intravenously administered in acute cases when the oxygen transport has been suddenly interrupted by the occlusion of a primary artery, such as myocardial infarct, stroke or cerebral infarct, occlusion of femoral artery. In fact in these conditions the oxygen deficiency at the diseased tissues is immediate and there is a request for a supplementary oxygen supply.

When used intraarterially or intravenously, liquids enriched with oxygen under pressure according to an embodiment of the invention, are administered using a cannula and a system for transfusional applications. These treatments are usually performed on the terminal artery of the organ or tissue in need of treatment. The same applies in the case of the treatment of ulcer-varicose lesions or pelvic inflammatory processes.

In accordance with another embodiment, the oxygen enriched solutions made according to the method of the invention can be locally administered in the cases where the demand for oxygen is increased such as in injuries at the epidermis in cases of burns, or local inflammatory processes or in case of frostbites or in gynecology for example for sterility treatment and in traumatology for example in cases of bone fractures and damages of soft tissues.

In accordance to another embodiment, the degenerative changes connected to biliary or liver system can be treated with the oxygen enriched solutions of the invention. With the oxygen treatment the zones of local hypoxya that are a consequence of degenerative changes, are reduced and the damaged tissues regenerates faster. For example, in cases of negative effects of radiotherapy, which are characterized by a vascular net deficiency and blood vessels rigidity causing lowering of blood flow, the infusion of an solution of an enriched physiological solution of the invention permit to a reparation of the tissues damaged by irradiation, lower the connected pain and expands the movement range.

In accordance with yet another embodiment an oxygen enriched liquid of the invention is suitable to treat or prevent the infectious diseases caused by viruses or bacteria such as the spore-forming Bacillus anthracis by the topical or endovenous administration of a antibacterial effective amount of a physiological oxygen enriched solution made according to the invention. Advantageously, said oxygen enriched solution can also incorporate antibiotics or other anti-infective agents suitable against bacterial infection. In these cases the administration of the solution is advantageously made twice a days for a period of time necessary to wipe out the infection.

The oxygen enriched solutions of the invention were tested on experimental animals subjected to an acute occlusion of blood vessels which provoked degenerative changes of the interested tissues.

An oxygen enriched physiological solution of 0.3 cc having an oxygen partial pressure of 2 bar was intravenously administered at a rate of 1 g/min twice a days to a first group of 5 rats having a myocardial infarction due to a restriction of the coronaries. A second group of rats with the same disease, was subjected to a conventional oxygen treatment by inhalation by means of a cannula.

The results of the treatment were monitored and scheduled for five days. The first group of animals, treated by administering a solution in accordance with an embodiment of the present invention consumed 20% of oxygen in the damaged tissues whereas the second group which received the oxygen by inhalation consumed only 5% of oxygen in the damaged tissues.

In accordance with another aspect of the present invention a cosmetic treatment is provided comprising the application of a cosmetically effective amount of an oxygen enriched liquid of the invention or a composition including said liquid.

In particular the local or subcutaneous application on of a solution or a cosmetic preparation including a cosmetically amount of a solution of the invention, provides an additional source of oxygen to the tissues in need of treatment, allowing a regeneration of damaged cells or tissues. In accordance with this aspect of the invention it is possible to treat facial corrugations, wrinkles and other signs due to skin aging by the local or topical application of an oxygen enriched solution or of a cosmetic preparation including such composition.

For example, the enriched solution of the invention can be incorporated in a solid cosmetic vehicle or in liposomes to provide emulsions, creams or ointments for local applications one or twice a day. These cosmetic preparations can further include cosmetic active ingredients which act in conjunction with the oxygen source.

In accordance with another aspect of the present invention an oxygen enriched water is provided which can be used as a beverage in spite of potable water or as a supplement in a diet regimen which requires an additional source of oxygen supply such as in sportsmanlike activities.

In these cases the administration of 1 liter per day in the normal dietetic regiment contributes to maintain an high efficiency of the principal physiological functions.

### BRIEF DESCRIPTION OF THE DRAWINGS

The method of the present invention together with additional objects and advantages thereof will be best understood from the following description of a specific, non limitative embodiment, when read in conjunction with the accompanying drawings in which:

FIG. 1 is a side view of an hyperbaric apparatus for the liquid enrichment with oxygen according to an embodiment of the invention;

FIG. 2 is a lateral view taken from the other side of the hyperbaric apparatus shown in FIG. 1.

### DETAILED DESCRIPTION OF THE INVENTION

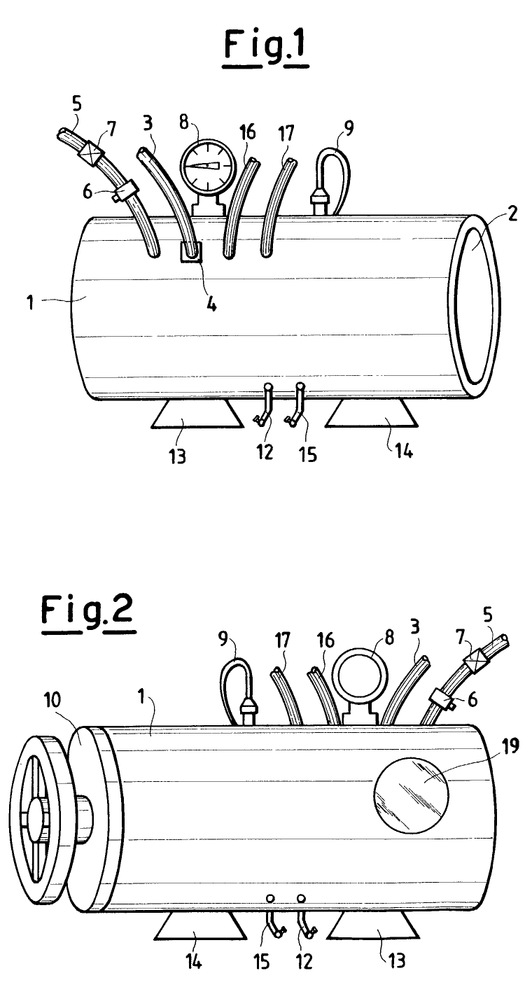

Referring now to the drawings and first, particularly to FIG. 1 thereof; with the generic number 1 is designed a cylindrical oxygen enrichment apparatus supported by two stands 13 and 14 and extending horizontally according to an embodiment of the invention.

A liquid to be treated according to the invention is lead at atmospheric pressure to an oxygen enrichment chamber 2 of a hyperbaric apparatus 1 through a supply tube 3 connected with a supply tank, not shown. The liquid is supplied by means of a pump, not shown, into the enrichment chamber 2 and the flow of liquid is controlled by means of an inlet valve 4. Said liquid can be water or physiological solutions or other water-based solutions which optionally are subjected to a preliminary purification and/or filtration.

Specifically, for medical uses the water to be treated according to an embodiment of the invention is disinfected or sterilized before being subjected to the oxygenation treatment to avoid the use of bacterial or viral contamination.

The enrichment chamber 2, which is conveniently made of stainless steel, is hermetically sealed during operative conditions and is engineered to support pressures higher than 100 atmospheres, in order to avoid the outflow of gases during oxygenation procedures or explosions.

An oxygen supplying tube 5, suitably provided with a check valve 6 is joined to the enrichment chamber 2 for supplying an oxygen flow within the chamber 2. An Additional inlet 16 for supplying additional gasses such as nitrogen, air or hydrogen is also provided in proximity of said supply tube 3.

The oxygen flow which is introduced into the chamber 2, can be obtained from many convenient sources and is stored in a conventional oxygen vessel positioned in proximity of the chamber. The oxygen flow introduced into the chamber 2 is regulated and controlled by the inlet valve 6 which permits to avoid a dangerous overpressure.

Suitably, a flow meter 7 can also be provided in connection with said oxygen supplying tube 5 to allow precise monitoring of the continuous inflow of the oxygen.

A manometer 8 is also provided as additional device to relieve and monitoring the interior pressure of the chamber during the procedure of liquid oxygenation. Suitably, the apparatus 1 is also provided with a safety valve 9 to avoid that the pressure within the chamber 2, raises over dangerous levels.

As illustrated in FIG. 2, in accordance to an embodiment of the invention a window 19 made of glass resistant to high temperature can be provided on the lateral side of the hyperbaric apparatus 1.

Conveniently, a door 10 driven in by a bayonet can also be provided on a lateral surface of the apparatus 1 to permit inspections of the internal chamber 2 and to allows specialized personal to enter the hyperbaric chamber for the periodic mechanical cleaning of inner surfaces thereof. Suitably, the internal chamber 2 is smooth-surfaced to allow an easy mechanical and chemical sterilization of its inner surface.

Suitably, the amount of water supplied in the hyperbaric chamber 2 does not exceed a third of the internal volume thereof. Conveniently, when the amount of supplied water reaches the desired volume, the inlet valve 6 is opened and an oxygen flow under pressure of about 11 absolute atmospheres (11 bar) is introduced into the oxygen enrichment chamber 2. The oxygen is supplied until the internal oxygen pressure is of about 11 atmospheres. These conditions are maintained for a time of about 60 minutes during which the internal pressure is constantly metered by the manometer 8.

During the process of water oxygen enrichment performed at hyperbaric conditions, it is also possible to check the rate of oxygen diffusion into the water by means of the manometer 8 and to control the status of the treatment by means of the window 19.

After the pressurization time of about 60 minutes a sufficiently high degree of oxygen enrichment of water is achieved and the enriched water can be recovered after the chamber has been depressurized by flowing the oxygen atmosphere through the exhaust tube 17. After leaving the enrichment chamber 2, the oxygen enriched water is forwarded through the outlets 12 and 15 to a bottling plant or to a unit storage not shown.

When the oxygenated water is stored in a bottle or in a sealed container, the retention of the high level of oxygen is retained for prolonged times. This means that when the bottles or containers are opened and the enriched water consumed, the water retains close to its initial oxygen content during its consumption. Advantageously, before said bottles or container are sealed the partial oxygen pressure is measured and the values should be the same as they were at the end of the oxygen enrichment treatment performed inside the hyperbaric chamber 2.

In accordance with a preferred embodiment of the method of the invention, physiological sterilized solutions, that are kept in closed containers such as vials for intravenous or intraarterial administration, can be prepared and used for oxygenation purposes.

1. A method for enriching a liquid with oxygen comprising the steps of: introducing a liquid into an oxygen enriching chamber of a sealed hyperbaric apparatus;
introducing an oxygen flow under pressure into said enriching chamber;
subjecting said liquid to an oxygen pressure to enrich said liquid with oxygen;
recovering the oxygen enriched liquid.

2. A method according to claim 1, wherein said liquid is subjected to an oxygen pressure of from 8 to 14 atmospheres within said enriching chamber.

3. A method according to claim 2, wherein the oxygen pressure of from 8 to 14 atmosphere is maintained within said enrichment chamber for a time of 45 to 70 minutes to achieve a stable oxygen enriched liquid.

4. A method according to claim 1, wherein the liquid is maintained under conditions of high pressure to reach an oxygen concentration of 1-3 ml O₂/g in the liquid.

5. A method for preparing an oxygen enriched liquid comprising the steps of:
contacting a liquid with oxygen under hyperbaric conditions within an oxygen enrichment chamber of a sealed pressurization apparatus;
maintaining said liquid under oxygen hyperbaric pressure to dissolve the oxygen in the liquid;
passing the oxygen enriched liquid throughout capillary tubes at a speed to eliminate the generation of bubbles from the liquid; and
recovering the liquid enriched with oxygen.

6. A method according to claim 5, wherein the oxygen is dissolved in the liquid under a partial pressure of 3 to 10 Mpa.

7. A method according to claim 5, wherein said liquid is maintained into contact with oxygen under hyperbaric conditions until a the concentration of oxygen in the liquid is of from 1 to 3 ml O₂/g.

8. A method according to claim 5, wherein said capillary tubes in which the oxygen enriched liquid is passed have a diameter of 60 to 80μ.

9. A method according to claim 5, wherein the oxygen enriched liquid is passed thorough capillary tubes under hydrostatic pressure of 50 to 70 Mpa.

10. A method according to claim 5 wherein the oxygen enriched liquid transits in said capillary tubes at a speed of 4 m/s.

11. A method according to claim 5 wherein said oxygen enriched liquid is passed thorough the capillary tubes at a rate to obtain a transit of 1 g/min.

12. A method according to claim 5 wherein said liquid is drinkable water.

13. A method according to claim 5 wherein said liquid is a solution selected from the group consisting of physiological solution, dextrose solution, Ringer solution, Hartmann solution protein rich solution PVP plasma and mixtures thereof.

14. A drinkable oxygen enriched water obtainable according to the process of claim 1.

15. A drinkable oxygen enriched water obtainable according to the process of claim 5.

16. A method of treatment of conditions connected with a deficiency of oxygen supply comprising the administration to a human of an effective amount of an oxygen enriched liquid prepared according to the method of claim 5.

17. A cosmetic method of treatment comprising the local application of a cosmetically effective amount of a cosmetic composition including an oxygen enriched liquid prepared according to the method of claim 1.

18. A method of treatment bacterial infections comprising the administration of an antibacterial effective amount of a physiological enriched solution prepared according to the method of claim 5.
